(19) United States
(12) Patent Application Publication
(10) Pub. No.: US 2015/0257705 A1
(43) Pub. Date: Sep. 17, 2015

(54) Catheter for Measuring the Blood Flow of a Body Tissue

(71) Applicant: CARAG AG, Baar (CH)

(72) Inventors: Kuno Limacher, Steinhausen (CH); Jerome Bernhard, Zurich (CH); Jorg Hummen, Kilchberg (CH); Jevgenij Mannhart, Cham (CH); Claudio Steiner, Schwyz (CH); Cyrill Rothlin, Hunenberg (CH); Daniel Napoletano, Eglisau (CH); Daniel Uhr, Baar (CH); Daniel Schenk, Affoltern am Albis (CH)

(21) Appl. No.: 14/725,802

(22) Filed: May 29, 2015

Related U.S. Application Data
(63) Continuation-in-part of application No. 13/057,579, filed on Feb. 8, 2011, now Pat. No. 9,066,693.

(30) Foreign Application Priority Data
Aug. 6, 2008 (CH) 01229/08

(51) Int. Cl. A61B 5/00; A61M 25/00; A61M 25/06; A61B 5/026; A61B 5/0205

(52) U.S. Cl. CPC A61B 5/6852; A61B 5/0261; A61B 5/02055; A61M 25/0662; A61M 25/007; A61B 5/01

(57) ABSTRACT

A catheter for measuring a flow of blood through a body tissue comprises a light emitter for emitting light into the body tissue and at least one light receiver for receiving light reflected in the body tissue and back to the catheter. The light emitter and the at least one light receiver are arranged in the catheter, wherein the catheter comprises a middle piece and a first and a second connection tube. The middle piece has a distal fastening piece for fastening to the first connection tube and a proximal fastening piece for fastening to the second connection tube, wherein the light emitter and the at least one light receiver are arranged in the middle piece.

### CROSS REFERENCE TO RELATED APPLICATIONS

The present application is a continuation-in-part of U.S. patent application Ser. No. 13/057,579, filed Feb. 8, 2011, which is a U.S. National Phase Application pursuant to 35 U.S.C. §371 of International Application No. PCT/CH2009/000241 filed Jul. 7, 2009, which claims priority to Swiss Patent Application No. 1229/08 filed on Aug. 6, 2008. The entire disclosure content of these applications are herewith incorporated by reference into the present application.

### FIELD OF THE INVENTION

The present invention relates to a catheter, a catheter system and a tube for measuring the flow of blood through a body tissue, particularly through a cerebral tissue. The invention furthermore relates to a catheter for cerebral diagnostics and/or therapy.

### BACKGROUND

Various devices for measuring the flow of blood through a body tissue are known from the prior art. To measure the flow of blood through the cerebral tissue of the brain, catheter-type measurement devices are used which carry measurement sensors at their catheter tip. Such catheter probes are inserted into the cerebral tissue though an opening made in the cranium, in order to carry out a measurement of the flow of blood through said tissue. A number of measurement methods are known for this purpose, for example thermal diffusion, ultrasound methods, and near-infrared spectroscopy in association with an indicator.

For example, EP 1 464 276 A1 discloses a measurement device for determining the flow of blood, in which device two octodes are placed on the surface of the head at a distance from each other. One of the octodes is connected to a radiation source which emits radiation with a near-infrared wavelength. Some of the radiation reflected on the cerebral tissue strikes the second octode, such that the intensity can be determined by an evaluation unit. Indocyanine green is used as the indicator, and a beam of light at a wavelength of between 780 and 910 nm is used. In this method of performing a near-infrared spectroscopy measurement, account must be taken of a large number of external influences that have a disadvantageous impact on the measurement of the flow of blood. The beam of light cannot be conveyed directly to the tissue that is to be measured, and instead it first has to pass through the skin, the skull cap, the dura mater, etc., in order to reach the tissue that is to be examined. As a result, the measurement signal is weakened and distorted by absorption and scattering, for example. It is therefore only possible to measure areas of tissue near to the surface of the head. Areas in the interior of the brain, for example near the floor of the ventricle, can be examined only with inadequate precision by this method.

EP 1 504 715 discloses a catheter with a light-emitting optical conductor and a light-receiving optical conductor, the ends of which optical conductors are arranged at a predetermined distance from each other.

Another device for measuring the flow of blood within the cranium is known from U.S. Pat. No. 5,579,774, for example. A catheter probe, inserted into the interior of the brain, comprises a measurement sensor for carrying out laser Doppler flowmetry. The beam of light of a helium-neon laser at 632.8 nm is guided in the axial direction via a light conductor to the measurement area, which lies distally in a continuation of the probe. Some of the incident light is absorbed and reflected by the surrounding tissue and some by the circulating blood. The reflected light is guided through at least one optical fibre to a processing unit. The light reflected from the moving red blood cells undergoes a Doppler shift, from which it is possible to determine the flow rate. The head of the catheter probe has a rounded tip whose diameter widens conically in the distal direction. The blunt tip is pushed through the opening in the cranium and through the subjacent tissue as far as the measurement area lying within the brain. The whole surface of the blunt tip presses against the cerebral tissue and exerts a pressure that can leave behind permanent damage in the brain.

### SUMMARY

According to one aspect of the invention a device is made available which measures the flow of blood through a body tissue, in particular through deep-lying cerebral tissue, and which is easy and inexpensive to produce and does not distort the measurement.

According to another aspect of the invention a device is made available which measures the flow of blood through a body tissue, in particular through deep-lying cerebral tissue, and which has the narrowest possible configuration and permits insertion of the device with minimal trauma, i.e. with minimal injury, into cerebral tissue.

According to another aspect of the invention a device is made available which measures the flow of blood through a body tissue, in particular through deep-lying cerebral tissue, and which permits insertion of the measurement device with minimal trauma into cerebral tissue.

According to another aspect of the invention a device is made available which measures the flow of blood through a body tissue, in particular through deep-lying cerebral tissue, and which permits reliable measurement of the flow of blood.

According to another aspect of the invention a device is made available which measures the flow of blood through a body tissue, in particular through deep-lying cerebral tissue, and which is transportable along with a patient.

In a preferred embodiment of the catheter according to the invention, a single optical fibre is present as optical conductor for the light to be emitted and for the reflected light, and a photodetector and a drainage channel for drainage are present.

In another preferred embodiment of the catheter according to the invention, a single optical fibre is present as optical conductor for the light to be emitted, as are a photodetector in the catheter for the reflected light, and a drainage channel for drainage.

In another preferred embodiment of the catheter according to the invention, the above-described head part is present, as are a pressure sensor and a drainage channel for drainage.

Said drainage channel can additionally or alternatively be used as a guide channel. In a catheter made of a largely flexible material, a stiff wire can be pulled through the guide channel, making it easier to insert the catheter into the body tissue.

A preferred embodiment of the invention is a device for measuring the flow of blood through a body tissue comprises a catheter with a catheter head for insertion into the interior of a body tissue, an optical conductor inside the catheter, a light source for emitting a beam of light into the body tissue by means of the optical conductor, and a processing unit for determining the rate of blood flow by means of a beam of light reflected from the body tissue. According to the invention, the catheter has a middle piece with a recess, a cutting, an aperture or an indentation which is oriented inwards into the middle piece and is provided laterally with respect to the longitudinal axis of the catheter. The recess or the like can have a round, oval or parabola-shaped inner surface or, in the case of an indentation for example, can be made up of a plurality of walls. According to the invention, the recess or the like has a surface area from which the optical conductor opens out and from which the beam of light thus emerges, and a further surface area which lies opposite the surface area from which the optical conductor opens out and which is oriented at least partially obliquely with respect to the axis of the optical conductor and preferably also obliquely with respect to the longitudinal axis of the catheter. For simplicity, the two surface areas are referred to herein below as the light exit surface or light exit surface area and as the reflection surface or reflection surface area. The optical conductor emerges from the light exit surface in such a way that the beam of light emitted from the light source is directed to the reflection surface and is deflected by the latter into the surrounding body tissue. The beam of light is absorbed and reflected in the body tissue in a manner characteristic of the flow of blood, as a result of which a reflection light beam is formed which is reflected on the reflection surface and coupled into the optical conductor. The reflection light beam is preferably focussed on the reflection surface. The reflection light beam is transmitted through the optical conductor to the processing unit, where the rate of blood flow can be determined from this delivered signal by comparison with the input signal according to the emitted beam of light.

The catheter thus forms a probe or catheter probe for measuring the flow of blood through the body tissue. The catheter has a specially designed catheter head, as is explained further below, as a result of which it is especially suitable for measurements on cerebral tissue, particularly on deep-lying cerebral tissue. The optical conductor can be formed, for example, by a fibre-optic cable or another light conductor, which is routed through the catheter to the middle piece and to the light exit surface of the latter. The reflection surface can itself have a surface roughness that is of sufficient quality, preferably of mirror-like quality, to allow it to reflect the beam of light from the light conductor. Preferably, a reflector, for example a mirror, is arranged on the oblique surface so as to reflect the beam of light into the surrounding body tissue. It is also advantageous if the reflection surface area has a curved shape in order, on the one hand, to reflect the incident beam of light into the tissue in a focussed manner and, on the other hand, to ensure that the light reflected on the tissue is fed into the optical conductor. In the configuration of the recess with its associated surface areas, the distance between the exit point of the emitted beam of light from the optical conductor and the opposite reflection surface is known and is provided such that the optical conductor focuses the emitted beam on the focal point of the reflection surface if the latter has a curved shape. The emitted beam of light is preferably reflected at an angle of 45° into the surrounding tissue. However, other angles of reflection are also conceivable, e.g. in a range of 30°-60°.

The recess is preferably filled or sealed or plugged with a material transparent to light. The outer surface of the filler material is preferably flush with the circumferential surface of the surrounding area, such that a smooth transition is obtained. Epoxy resin, for example, can be used as filler material. By filling the recess, it is possible to avoid air inclusions in the beam path.

It is preferable to use a light source with coherent light, e.g. a laser or a laser diode, which can emit light in the near infrared range between 780 and 910 nm. Light in this wavelength range is able to penetrate biological tissue. It is preferable to specifically use wavelengths that are suitable for a chosen measurement technique. Light with wavelengths of 785 nm, 850 nm and 905 nm is preferably used, which in particular is absorbed and reflected by oxygenated and deoxygenated haemoglobin and the marker substance indocyanine green. It is also advantageous to use a pulsed light beam or pulsed light beams of variable frequency. The parts of the incident beam of light reflected on the tissue are transformed in the processing unit by means of an analog-digital converter into a meaningful signal relating to the time profile of the presence of oxygenated and deoxygenated blood and of the marker substance.

It is also possible, however, to use a light source that emits light over a broad wavelength spectrum, for example a source of white light. The relevant wavelength ranges for measurement of the flow of blood can then be detected by a spectrometer. However, sources of coherent light have the advantage of requiring less energy.

The catheter probe is also suitable for tissue areas lying far within the brain. For this purpose, the distance between the catheter head and the middle piece is advantageously variable. A connection element, e.g. in the form of a tube, of greater or lesser length can be used depending on the desired measurement range or the desired depth of penetration into the brain. The catheter head can be inserted into the brain as far as the ventricle floor, for example, which then serves inter alia as reference point for locating the middle piece. The measurement of the flow of blood can thus be performed directly in situ in the areas of body tissue that are of interest.

The device for measuring the flow of blood through a body tissue according to the invention also preferably comprises a holder which is placed on the surface of the head, over an insertion opening in the cranium, and which holds the catheter probe in a predetermined position. The holder has, for example, a contact surface for placing onto the surface of the head and, protruding from this contact surface, a guide tube for the catheter. The holder can avoid penetration of infectious substances into the brain during a measurement. It acts as a barrier against pathogens and contaminants. The guide tube is preferably arranged perpendicularly on the contact surface, such that it guides the catheter head perpendicularly through the cranium and then holds it perpendicularly in position. In this position, the catheter probe can be turned about its axis without being deflected from its position and damaging surrounding tissue. By turning the probe, the attainable measurement field is greatly enlarged. A measurement can be performed in a 360° sector around the middle piece of the catheter probe.

The catheter probe can also have at least one X-ray marker, which can indicate the position and orientation of the probe in the tissue when an X-ray measurement is being carried out.

If the catheter head and the middle piece are made of metal, their position can be monitored by X-ray methods. In principle, however, other biocompatible materials are also conceivable, in which case the monitoring by X-ray methods can be achieved by opal additives, by electronic components already used in the catheter, or by markers. Moreover, the catheter can be provided with a scale for the depth of penetration into the body tissue and also with an angle scale for indicating the angle position of the middle piece. These features permit a precise locating of the measurement area in the brain.

At the same time, the catheter probe can be equipped with additional measurement sensors. For example, a temperature measurement sensor, e.g. a thermistor or a thermocoupler, can be provided in the middle piece. The temperature sensor can be arranged in an outer area of the middle piece, e.g. in a channel. Since metal has an excellent heat conduction coefficient, it is possible, at the same time as measuring the flow of blood through the tissue surrounding the middle piece, to also detect the temperature of the tissue. Contact with the tissue is not necessary in this case. However, if the middle piece is made of plastic, contact between measurement sensor and tissue must be ensured. The leads needed for the measurement can be routed through the catheter to the processing unit, in which the temperature signal is received and converted. However, it is also possible to arrange a temperature measurement sensor in a tube area between the tip of the probe and the middle piece.

Moreover, the catheter head and the middle piece of the catheter can be provided with a passage or a drainage channel that permits drainage of the surrounding tissue through the catheter. For this purpose, the catheter head, for example, has at least one opening which leads to the surrounding tissue and which is connected to the drainage channel, such that fluid can be aspirated from the tissue through the channel. Arranged near the drainage opening, there is preferably at least one pressure sensor which is designed, on the one hand, to determine the pressure in the surrounding tissue and, on the other hand, to determine the pressure in the drainage channel. In the event of an occlusion and a subsequent change of pressure in the drainage channel, a signal can be output to the processing unit, such that an alarm can be triggered. In principle, it is also possible to deliver liquid to the tissue by way of the drainage opening, i.e. to inject liquid.

According to a further aspect of the present invention, a catheter head for insertion into a body tissue is provided which is divided into an insertion area and an adjoining connection area. The insertion area comprises several apertures in its surface. Moreover, the insertion area has a diameter that increases in the direction of the connection area. The apertures are provided in the insertion area in such a way that webs extending in the direction of the connection area are formed between the apertures along the surface of the catheter head. The webs begin in an area of the insertion area with a small diameter and end in an area with a greater diameter.

This inventive design of the catheter head permits gentle and largely atraumatic insertion of the catheter head into and through the body tissue, particularly a cerebral tissue. During insertion of the catheter head, the tissue is initially spread apart only by the frontmost area and by the surface of the webs. The tissue is not yet stressed in the area of the apertures between the webs. After this initial spreading, the catheter head is inserted farther and the tissue areas at the apertures are also widened by the circumferential surface of the connection area. In this procedure, only minimal direct pressure is applied to the tissue.

The connection area of the catheter head is preferably circular or oval and has a uniform diameter. The diameter is preferably at most 3 mm. The insertion area is closed at the frontmost area of the tip and, for example, is parabola-shaped, round, or in the form of a truncated cone. The apertures can extend in an elongate shape from the insertion area to the connection area. Because of the increasing diameter of the insertion area, the webs between the apertures are curved in the longitudinal direction of the catheter. In a plan view of the tip, this results in a cross-shaped or star-shaped arrangement of the webs, depending on the number of apertures.

In one embodiment of the invention, the apertures have openings or are preferably formed completely by openings. The edges or boundaries of the apertures or openings are rounded or bevelled so as to provide a smooth transition of the adjoining surfaces. Inside it, the catheter head can have a channel which, together with a channel of the catheter, can form a drainage line in the proximal direction. The channel is connected to the openings that form the apertures. It is thus possible to use the openings as drainage openings for removal or delivery of fluid from or to the surrounding tissue. In order to form the drainage channel, a fastening piece suitable for fastening to a connecting pipe, preferably to a flexible tube part, of the catheter can adjoin the connection area in the longitudinal direction. The diameter of the connecting pipe or of the tube part on the fastening piece corresponds substantially to the diameter of the connection area, so as to give a smooth transition between these structural parts.

In a preferred embodiment, the recess consists of at least two windows. In a preferred embodiment these windows are located in an upper part of the middle piece, such as the lid.

The light emitter and light receiver can also be arranged in a stiff element, such as the tip element. The stiff element can also be the combination of the middle piece and the tip element being combined with each other without having a flexible tube element in between. The structure and layout of the catheter device as described herein however remains the same. This embodiment is therefore claimed as a separate invention as well.

In another embodiment, only one of the light emitter and the light receiver is arranged in the middle piece and the other one, preferably the light receiver, is arranged in the tip element. The first flexible tube part is preferably present between the tip element and middle piece. This enables to variably change the distance between the light emitter and the light receiver. The light receiver is preferably a photodetector. The structure and layout of the catheter device as described herein however remains the same. This embodiment is therefore claimed as a separate invention as well.

In a variant of the embodiments described above, more than one light receiver, preferably more than one photodetector, is present, wherein the light receivers are arranged at a distance to each other. They can all be arranged in the middle piece or in the tip element or some of them are arranged in the middle piece and some are arranged in the tip element.

In a preferred embodiment, a smooth transition of the structural parts of the device is enabled by using a second tube consisting of an inner and an outer tube, wherein the middle piece is attached to the outer tube only, the outer tube thereby protruding the inner tube. This kind of double tube can be used for other catheters and other kind of devices as well. For example, in another embodiment of the catheter device, the light emitter and light receiver are arranged in the tip element and no middle element is present. In still another embodiment, the middle piece and the tip element are combined with each other without having a flexible tube element in between. In both embodiments, the inventive tube as described above can be used as well. This combination of double tubes forming one single tube with a step in its one end or both ends is therefore claimed as a separate invention as well.

In a preferred embodiment, the catheter is covered with a protection sleeve. The sleeve covers the catheter along its length. The protection sleeve comprises a distal end which protrudes a distal end of the catheter. The protection sleeve is open at its distal end and extends at a distance to the catheter located within the sleeve, wherein the protection sleeve is not transparent at least for visible light. This facilitates zero setting of a sensor of the catheter, especially of a light sensitive pressure sensor. This kind of protection sleeve can be used with other catheters as well, such as catheters with no middle piece but with light sensitive sensors arranged in a tip element. This protection sleeve is therefore claimed as a separate invention as well.

More variants of the invention and additional inventive embodiments are described in the dependent claims. Note is made that combinations of the features of the different dependent claims can be made as well and are part of this disclosure in order to obtain additional embodiments not described in the following in detail.

### BRIEF DESCRIPTION OF THE FIGURES

An embodiment according to the present invention is shown in the drawings. Features of the measurement device that are evident from the figures are to be regarded as belonging to the scope of the disclosure and are not to be interpreted in any way as limiting the invention. In the drawings:

FIG. 1 shows an overall view of a measurement device according to the present invention;

FIG. 2 shows a three-dimensional view of a catheter head according to the present invention;

FIG. 3 shows a front view of the catheter head according to FIG. 2;

FIG. 4 shows a longitudinal section through the catheter head according to FIG. 3; and

FIG. 5 shows a three-dimensional view of a first embodiment of a middle piece of a measurement device according to the present invention;

FIG. 6a shows a longitudinal section through a measurement device according to FIG. 1 along a first longitudinal plane;

FIG. 6b shows a longitudinal section through a measurement device according to FIG. 1 along a second longitudinal plane perpendicular to the first plane;

FIG. 7 shows a three-dimensional view of a second embodiment of a middle piece of a measurement device according to the present invention;

FIG. 8 shows a longitudinal section through a measurement device from FIG. 1 according to the second embodiment;

FIG. 9 shows a perspective view of a middle piece according to the invention;

FIG. 10 shows a perspective view of a lid of the middle piece according to FIG. 9;

FIG. 11 shows a view of the distal end of a lower part according to FIG. 9;

FIG. 12 shows a view of the proximal end of the lower part of the middle piece according to FIG. 9;

FIG. 13 shows a perspective view of the lower part according to FIG. 12;

FIG. 14 shows a top view of the lower part according to FIG. 12;

FIG. 15 shows a longitudinal section through the lower part according to FIG. 12 with optical conductor and circuit board;

FIG. 16 shows a schematic view of the measurement device according to the invention;

FIG. 17 shows a three-dimensional view of a catheter head according to the present invention, in another embodiment;

FIG. 18 shows a measurement device according to a second embodiment of the invention;

FIG. 19 shows an enlarged perspective view of a part of the embodiment according to FIG. 18;

FIG. 20 shows the measurement device according to FIG. 19 in an exploded view;

FIG. 21 shows a longitudinal section of a middle piece according to FIG. 19;

FIG. 22 shows an enlarged longitudinal sectional view of a proximal end of the measurement device according to FIG. 18;

FIG. 23 shows an enlarged longitudinal sectional view of the middle piece of the measurement device according to FIG. 18;

FIG. 24 shows an longitudinal enlarged sectional view of a distal end of the measurement device according to FIG. 18;

FIG. 25 shows a side view of a part of the measurement device according to FIG. 18;

FIG. 26 shows a cross-sectional view along the plane A-A of FIG. 25;

FIG. 27 shows a cross-sectional view along the plane B-B of FIG. 25;

FIG. 28 shows a cross-sectional view along the plane C-C of FIG. 25;

FIG. 29 shows a perspective view of a lower part of the middle piece according to FIG. 20;

FIG. 30 shows a perspective view of an upper part of the middle piece according to FIG. 20,

FIG. 31 shows a perspective view of the middle piece of FIG. 20;

FIG. 32 shows a side view of the middle piece of FIG. 31;

FIG. 33 shows an enlarged cross-section of a second tube part and the middle piece;

FIG. 34 shows the measurement device of FIG. 18 with a protection sleeve;

FIG. 35 shows a longitudinal sectional view of the device and the sleeve of FIG. 34;

FIG. 36 shows an enlarged view of part X of FIG. 35;

FIG. 37 shows an enlarged view of part Y of FIG. 35 and

FIG. 38 shows a cross-sectional view along B-B of FIG. 35.

### DETAILED DESCRIPTION

In the following description, the end with the catheter tip is designated as the distal end of the catheter probe, and the opposite end is designated as the proximal end.

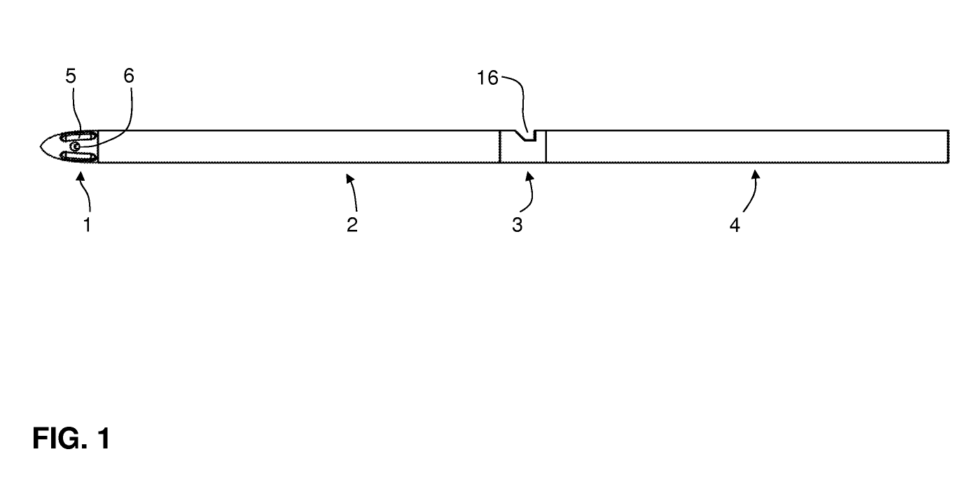

FIG. 1 shows a measurement device according to the present invention which has a tip element 1 as catheter head, a first connection tube 2, a middle piece 3, and a second connection tube 4. The first and second connection tubes 2, 4 are each preferably made from a flexible tube part. The text below therefore refers to a tube part, although this is also understood to mean other types of connection tubes 2, 4 that permit insertion of the catheter into the body tissue.

The tip element 1 is provided at the distal end of the measurement device. At the proximal end, the measurement device has a plug connector (not shown) or is guided directly into a processing unit for conversion and evaluation of the measurement signals. The tip element has several elongate openings 5, which form apertures in the surface of the catheter head. The openings 5 are provided for drainage of fluid in the surrounding tissue. Moreover, a round opening 6, in which a pressure sensor can be arranged, is provided between two elongate openings 5. However, the pressure sensor can also be arranged adjacent to, but not between, these irrigation openings 5. Electronic or optomechanical means, for example a silicon micromembrane, can be used as pressure sensors. The middle piece 3 has an indentation or aperture 16 into which a light conductor opens and through which light, delivered through the light conductor, can be emitted into the surrounding medium. The first tube part 2 can be varied in length depending on the intended nature of use of the measurement device.

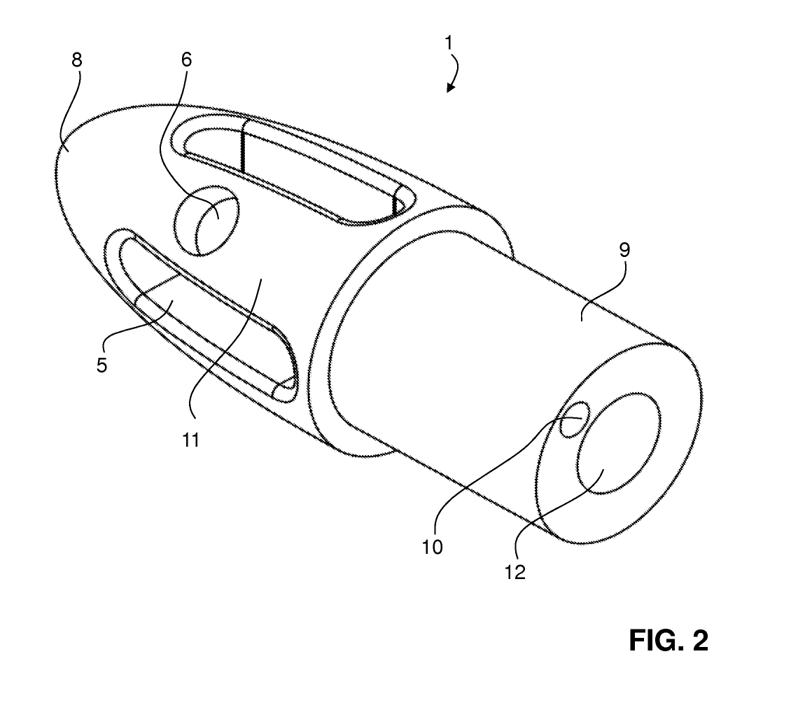

FIG. 2 shows a three-dimensional view of the catheter head in the form of the tip element 1. The tip element can be made of plastic or of metal. The tip element is divided into a distal insertion area and an adjoining connection area. In the insertion area, it has an oval, parabola-shaped or round tip 8. The insertion area has a diameter increasing from the tip 8 in the direction of the connection area. The tip 8 is closed at its centre. It is followed shortly thereafter by one or more elongate apertures or openings 5, which are arranged alongside one another in the circumferential direction. The embodiment shown in FIG. 2 has four openings, of which two can be seen in FIG. 2. Between the elongate openings 5, webs 11 are formed in the circumferential area and extend from the connection area into the tapering area of the tip 8 and are thus slightly curved. The edge areas of the elongate openings 5 are rounded or trimmed, so as to give a gentle transition from the circumferential surface of the catheter head to the edge areas of the openings 5. The slight curvature of the webs means that, in the tip area, they run towards the centre or radially towards one another. The space between two adjacent elongate openings 5, which space forms the webs 11, is approximately as wide as the width of one elongate opening. Because of the slight curvature of the webs, this circumferential area tapers in the direction of the tip.

At the proximal end, the tip element 1 has a fastening piece 9, which is adjoined by the first tube part 2. The fastening piece 9 is part of the connection area or adjoins the latter. The fastening piece 9 is designed like a sleeve. A tubular structure can be placed with a form fit over the circumference of the fastening piece 9. In the circumferential wall of the fastening piece 9, a channel 10 extends in the longitudinal direction all the way from the distal end into the insertion area. The channel 10 serves for the passage of pressure measurement elements. For example, electrical or optical leads can be routed through the channel 10 as far as the opening 6, in which a pressure sensor can be arranged.

The elongate openings 5 serve for the drainage of the surrounding tissue. The drainage fluid is conveyed through an axially extending channel 12, which extends into the insertion area of the catheter head. It is an advantage of the present invention that the pressure sensor can be arranged between the elongate openings 5 and therefore carries out a pressure measurement at the same level in the surrounding tissue where a fluid can be discharged via the drainage opening.

If the tip and the middle piece are made of plastic, the tip preferably has an X-ray marking, so as to allow the positioning of the tip to be checked.

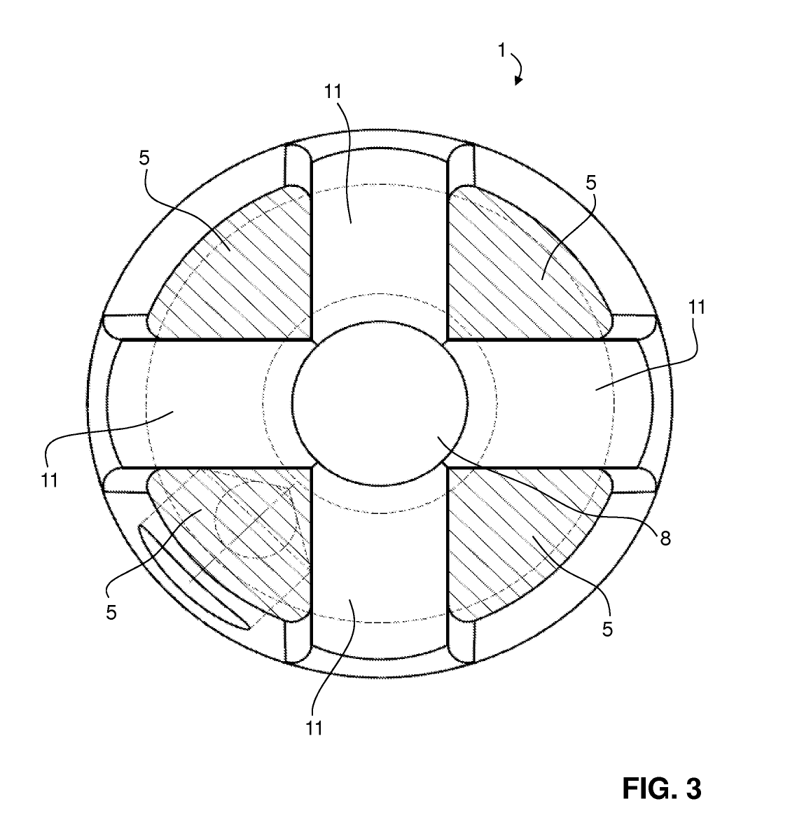

FIG. 3 shows a schematic front view of a tip element 1. At the centre, the tip 8 can be seen as the frontmost curve of the catheter head. Arranged around this centre are the four apertures in the form of elongate openings 5 which, in plan view, form a free space between each other. The circumferential areas between the elongate openings 5 form the webs 11, which extend from the centre of the tip 8 to the outer circumference of the connection area and form a kind of guide structure for the tip element 1 upon insertion into a cerebral tissue. The webs 11 appear in a cross-shaped arrangement in the plan view in FIG. 3. When the catheter head is inserted into the tissue, the tip 8, whose diameter is smaller than the diameter of the overall tip element, and the surface of the webs 11 are pressed directly onto the tissue. At first, no pressure is applied to the tissue in the area of the apertures, that is to say in the area of the elongate openings 5. The tissue is gently braced and spread apart by the webs 11, such that the catheter head can penetrate into the tissue and, in doing so, causes minimal trauma. It is only after the tissue in the insertion area of the tip element 1 has been initially widened by the webs 11 that the tissue is spread open completely across the entire diameter of the connection area. By means of this design of the catheter head of the measurement device, it is possible to insert the measurement device into interior areas of the brain virtually without injury.

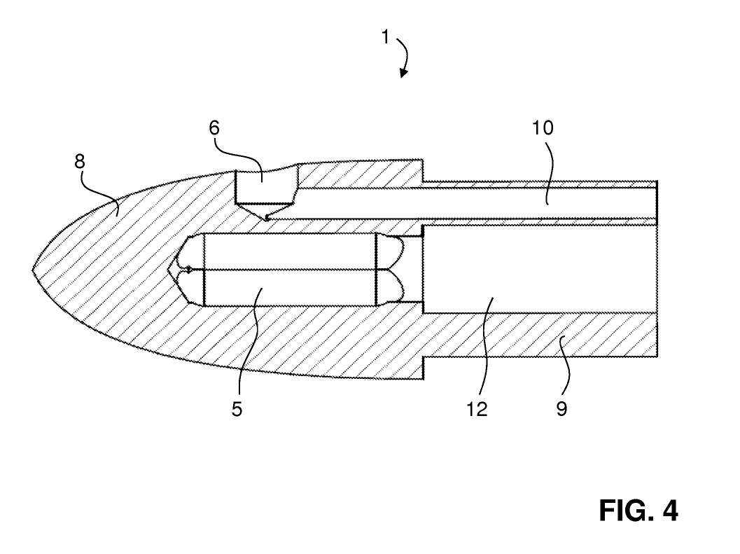

FIG. 4 shows a cross section along the longitudinal axis of a tip element 1. It can be clearly seen that the tip element 1 in the insertion area and the tip 8 has a smaller diameter, which increases in the proximal direction as far as the connection area, which is adjoined by the fastening piece 9. The channel 10 extends in a straight line in the longitudinal direction as far as the opening 6, which is provided for a pressure sensor. In the interior, the four elongate openings 5 are adjoined by a guide channel 12, which is provided for removal of drainage fluid. In this view, the frontmost area of the tip 8 is shown as being pointed, which ensures that the widening of the tissue is optimized while no damage can be caused by scratching or cutting. The tip 8, or the cross section through the webs 11 of the catheter head, is substantially parabola-shaped.

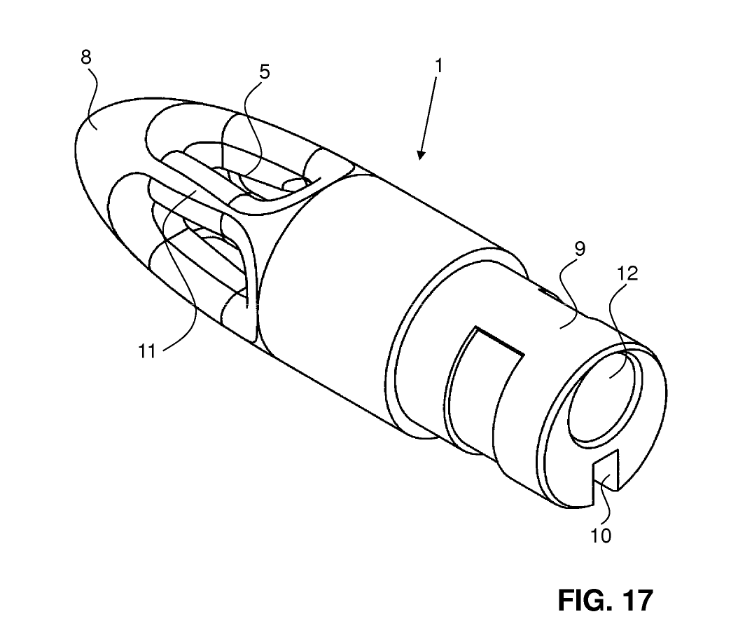

FIG. 17 shows an embodiment of a tip element 1 in an alternative to FIG. 2. Identical parts are provided with identical reference signs. The tip 8 is once again rounded. The openings 5 lie closer to one another than in the embodiment according to FIG. 2, and they are separated from one another only by short webs. The webs preferably have a width that is a multiple smaller than the width of the individual openings. The drainage channel 12 extends axially offset in relation to the longitudinal centre axis of the tip part 1. The pressure sensor channel 10, or the channel for other electrical lines, is in this case open. The pressure sensor is preferably arranged on that side of the openings 5 lying opposite the tip 8 or is located in the middle piece.

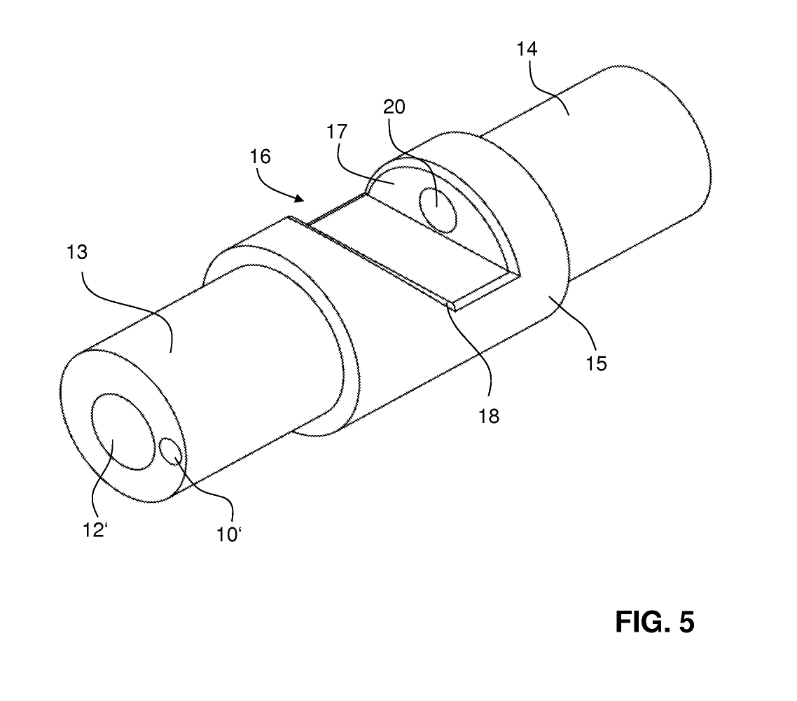

FIG. 5 shows a middle piece 3 according to the present invention. The middle piece 3 has a distal fastening piece 13 and a proximal fastening piece 14. The fastening pieces 13 and 14 are similar to the fastening piece 9. They are sleeve-shaped and have a central passage for a drainage channel 12′ for drainage of fluid and, in their circumferential wall, they have a continuous channel 10 for leads serving the pressure sensor in the tip element 1. The guide channel or drainage channel 12′ in the middle piece 3 is analogous to the channel 12 in the tip element 1 and forms a continuation thereof.

On the middle piece 3, a middle area 15 is formed between the proximal fastening piece 13 and the proximal fastening piece 14. In the embodiment according to FIG. 5, the middle area 15 has a wedge-shaped indentation 16 which, instead of extending all the way to the drainage channel 12′, leaves the latter closed. At its proximal end, the indentation 16 extends with a light exit surface 17, from which an optical conductor emerges, perpendicular to the longitudinal axis. At its distal end, the indentation 16 has a reflection surface 18, which preferably extends at a 45° angle to the longitudinal axis of the middle piece 3 and to the surface 17. A channel 20, which ends in the light exit surface 17 of the indentation 16, extends through the circumference of the distal fastening piece 14 and the proximal end of the middle piece 3, in the longitudinal direction of said middle piece 3. The opening of the channel 20 in the indentation 16 thus lies opposite the oblique surface 18. The channel 20 is provided for guiding a light conductor. The light conductor can also extend past the light exit surface and protrude into the indentation 16. The light from the light conductor strikes the opposite reflection surface 18. A mirror or other type of reflector, arranged on the reflection surface 18, reflects the light from the light conductor into the surrounding tissue around the middle piece 3. The reflector can be mounted as a separate element on the reflection surface 18, or the reflection surface 18 itself can be configured as a reflector surface. For example, a gold coating can be provided as reflection surface. It is also possible for the surface 18 or the reflector to have a slightly curved shape, such that the incident beam of light is widened slightly. The indentation 16 is filled with epoxy resin, such that the surface of the middle area 15 is cylindrical.

An individual optical fibre is preferably suitable as light conductor. There are preferably not more than five optical fibres present. Two or three fibres can likewise be used.

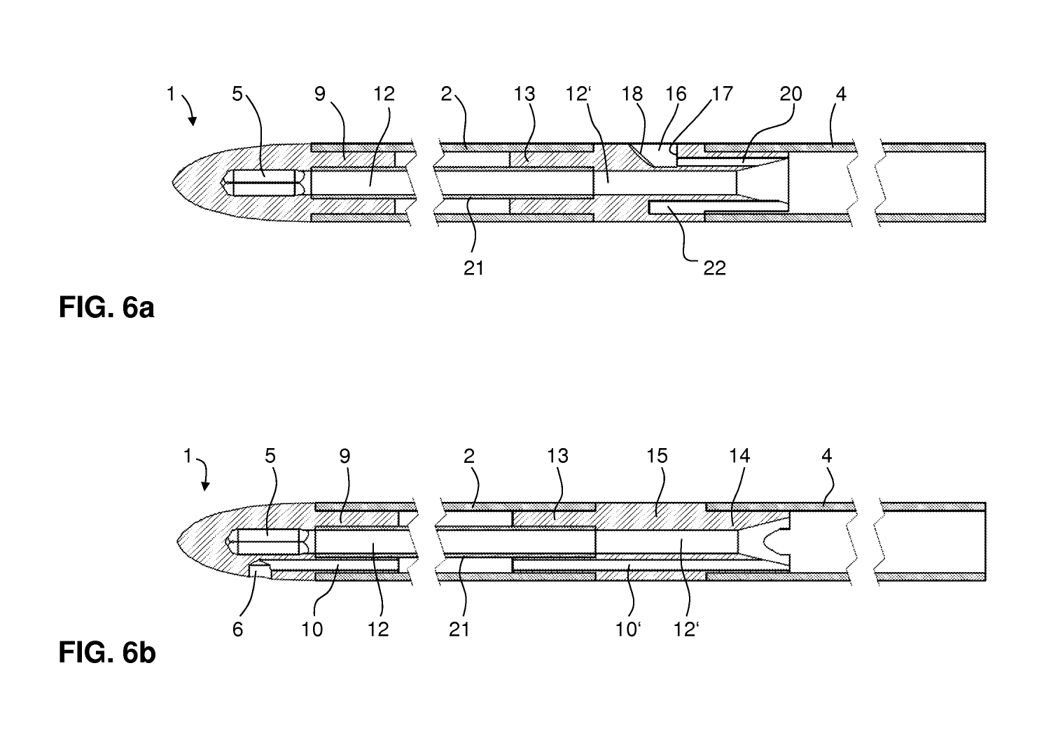

FIGS. 6a and 6b each show longitudinal sections through a first embodiment of a catheter probe according to the present invention, these longitudinal sections being offset by 90° to each other. FIG. 6a shows, from left to right, a tip element 1, a first tube part 2, a middle piece 3 and a second tube part 4 according to the view from FIG. 1. The first tube part 2 has one end pushed over the fastening piece 9 of the tip element 1 and has the other end pushed over the fastening piece 13 of the middle piece 3. In the interior of the first tube part, between the tip element 1 and the middle piece 3, there is another flexible tube 21 that interconnects the guide channels 12 and 12′ of the tip element and of the middle piece. The channels 12 and 12′ form, together with the tube 21, a drainage channel for fluid that is intended to be removed from the tissue surrounding the catheter tip, by way of the openings 5. The length of the tube parts 4 and 21 can be varied, such that the position of the middle piece 3 in the tissue can also be adjusted, i.e. tube parts of different lengths can be fitted between the tip element and the middle piece.

The wedge-shaped indentation 16 with the light exit surface 17 and the reflection surface 18 can be seen on the middle piece 3. The channel 20 opens into the light exit surface 17. The light exit surface 17 extends substantially perpendicular to the longitudinal axis of the catheter, although it could also be arranged at an angle thereto. An optical conductor, guided through the channel 20, is guided onwards through the second tube part 4 to a light source (not shown).

A temperature measurement channel 22 is also provided in the circumferential wall of the middle piece 3, on the proximal side, said temperature measurement channel 22 beginning on the side of the second tube part 4 and ending roughly at the centre of the middle piece 3. The temperature measurement channel 22 is provided for a temperature sensor that measures the temperature of the surrounding tissue. The temperature sensor is arranged at this point near the outer periphery of the catheter and is able to measure the temperature in the tissue without impediment or distortion. Leads (not shown) run from the temperature sensor through the second tube part 4 to the processing unit and transmit a temperature signal to the processing unit.

FIG. 6b shows, within the tip element 1, the pressure sensor channel 10 which extends from the proximal end of the tip element 1 as far as the opening 6. In the opening 6, there is a pressure sensor whose leads run through the channel 10 and the space between the first tube part 2 and the inner tube 21 as far as a channel 10′ in the middle piece 3. At the proximal end of the middle piece, the leads emerge from the channel 10′ and are routed further through the second tube part 4 as far as the processing unit, to which they transmit a signal corresponding to the pressure in the medium surrounding the tip element 1 or in the guide channel 12. The processing unit is able to control the drainage of fluid through the drainage channel depending on the pressure signal from the pressure sensor. Manual drainage can also be performed.

The channel 12′ on the middle piece 3 is widened conically at its distal end. This funnel-shaped opening facilitates insertion of a guide wire, which is used to guide the catheter probe during insertion into the body tissue.

The optical light conductor and the leads for the pressure sensor and temperature sensor can be routed freely within the interior of the second tube part 4. However, an inner tube can also be provided in this tube part, such that the leads are routed through the space between the outer tube and inner tube. The second tube part 4 can be adjoined by a plug in which the leads end. The plug can be fitted directly to the processing unit or to another plug, which in turn leads to the processing unit.

When examining a body tissue, for example the cerebral tissue in the interior of the brain, an opening is made in the cranium. A holder for the catheter is provided over the opening and holds the catheter as far as possible perpendicular to the surface of the head during insertion and also in the inserted state. Thus, the holder on the one hand covers the opening in the head and on the other hand ensures that the catheter is not accidentally deflected from its intended path during its insertion and during measurements. In this way, it is possible to avoid contamination of the area being examined and to avoid unnecessary damage to the surrounding tissue.

The catheter probe is inserted carefully through the tissue as far as a desired measurement position, for example until it rests on the floor of a ventricle. The inventive design of the catheter head means that in doing so there is only a minimal impact on the cerebral tissue, such that the tissue is not unnecessarily damaged and the measurement is not distorted.

To measure the flow of blood through the cerebral tissue, light in the near infrared range is conveyed through the light conductor in the channel 20 to the reflection surface 18. The light source can, for example, also be provided on the processing unit itself. The beam of light directed onto the surface 18 is reflected there and sent out into the cerebral tissue. There, it is absorbed and reflected among other things on the oxygenated and deoxygenated haemoglobin and, if appropriate, on the indocyanine green. Some of the reflected light falls back onto the reflection surface 18, as a result of which it is fed into the light conductor in the channel 20 by reflection on the surface 18. The channel guides the reflected light back to the processing unit, in which it is evaluated by, for example, an analog/digital converter. At the same time, the temperature in the measured tissue can be determined by the temperature sensor in the channel 22, and the pressure in the area surrounding the tip of the probe can be determined by the pressure sensor. If necessary, drainage can be performed immediately via the channel 12, 12′.

By means of the measurement probe being guided by the holder, the probe can be turned in its measurement position without thereby being deflected and damaging the surrounding tissue. By turning the probe, the measurement range in the tissue can be greatly widened. It is possible to cover a measurement field that extends through 360° around the middle piece 3. The tube parts between the tip and the middle piece can be flexible or rigid.

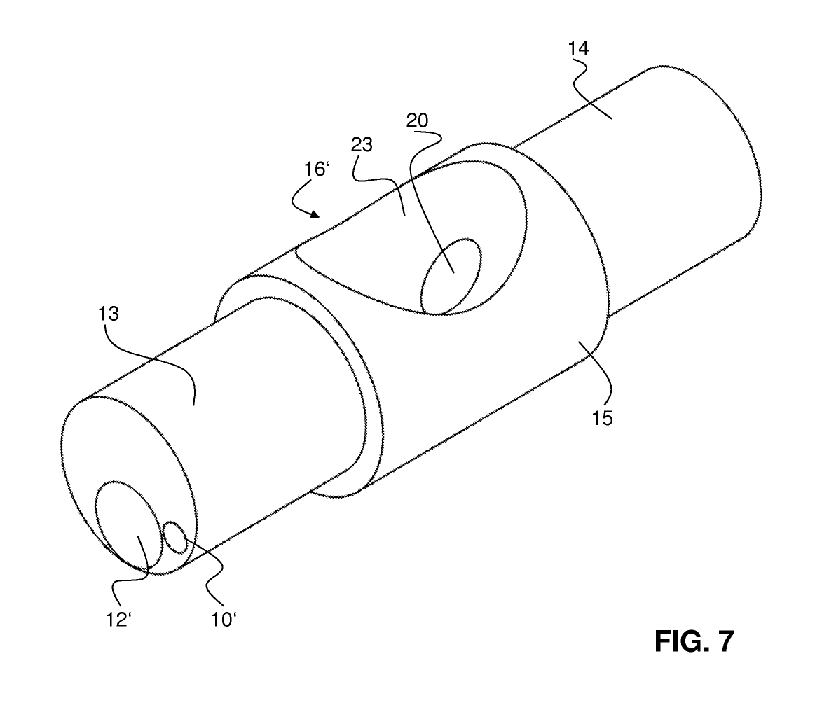

FIG. 7 shows a second embodiment of a middle piece according to the present invention. In this design of the middle piece, the drainage channel 12′ is eccentric to the longitudinal axis. In this way, the middle piece can be constructed with a smaller diameter. Moreover, the recess according to the invention is provided here in the form of an aperture 16′ which is sunk in a curved or rounded shape into the middle piece. An inner surface 23 thus formed comprises a light exit surface area at the proximal area, and, lying opposite this at the distal area, it has a reflection surface area. In terms of its function, the light exit surface area corresponds to the light exit surface 17 from the embodiment according to FIGS. 1 to 6, and the reflection surface area accordingly corresponds in terms of its function to the reflection surface 18. The light-conducting channel 20 opens out from the light exit surface area. Light emitted from an optical conductor in the light-conducting channel 20 strikes the opposite reflection surface area, from which it is reflected into the surrounding tissue. The curvature of the aperture 16′ is designed such that light reflected from the tissue is focussed into the optical conductor.

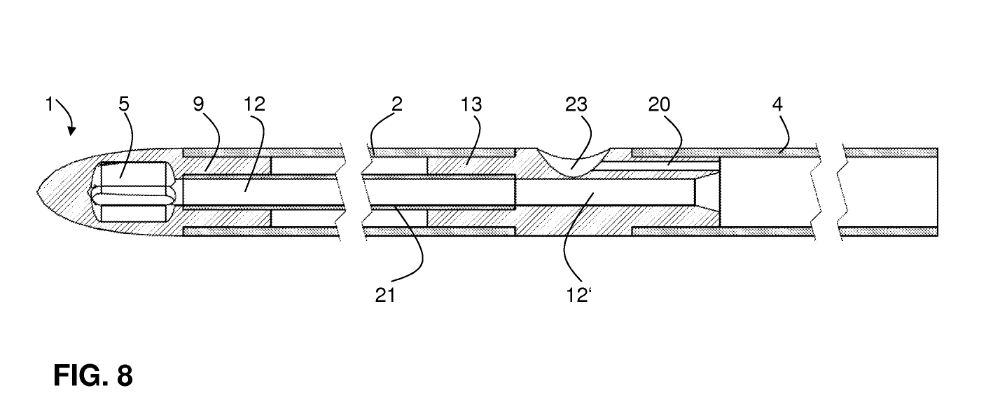

FIG. 8 shows a longitudinal section through a measurement device with a middle piece according to FIG. 7, but the drainage channel 12′ is arranged in a central position here. The aperture 16′ reaches almost as far as the drainage channel 12′ but leaves the latter closed. The curvature or roundness of the aperture 16′ is such that light from the channel 20 is oriented directly onto the focal point. The other elements of the measurement device in this embodiment correspond to those from FIGS. 6a and 6b.

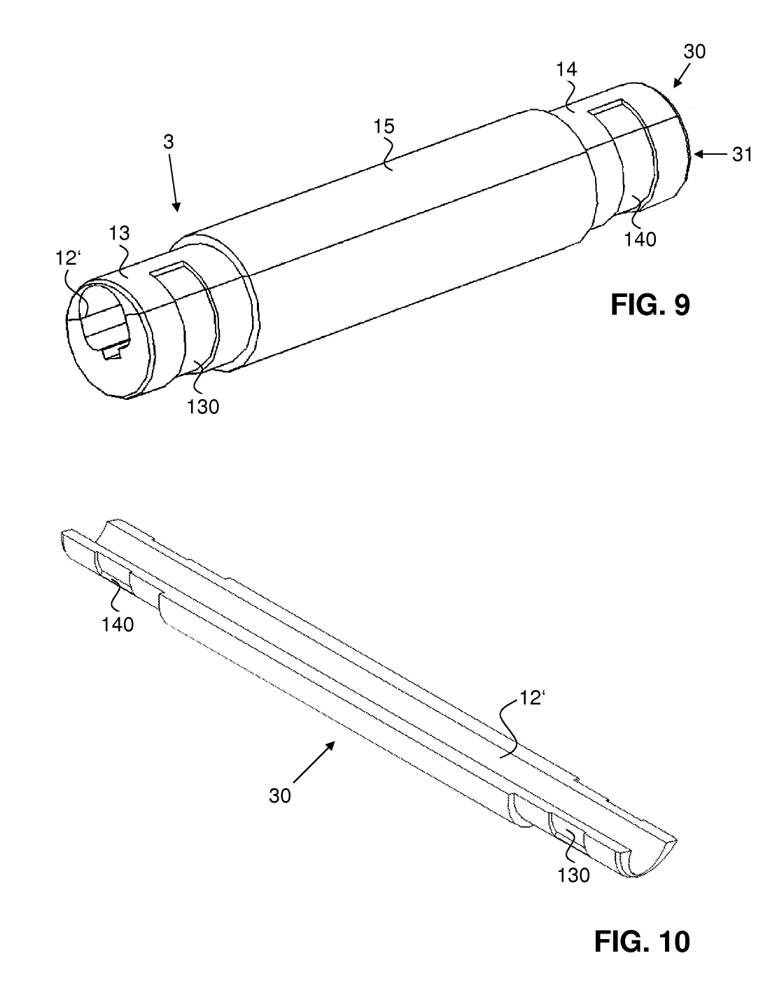

FIGS. 9 to 14 show the middle piece 3 according to the invention in a preferred embodiment. Identical parts are provided with the same reference signs as in the above examples. The middle piece 3 is made of plastic and is preferably produced by injection moulding. It is composed of at least two parts, namely a lower part 31 and a lid 30. Both are configured as half tubes, as can be seen in FIG. 10 for the lid 30. Together they form a tubing with a middle area 15, a distal fastening piece 13 and a proximal fastening piece 14. Grooves 130, 140 are provided extending radially about the circumference of the two fastening pieces 13, 14 in order to permit a connection to adjoining tubes. These grooves serve as adhesive grooves for a form fit and force fit. An equivalent groove can be seen in FIG. 17.

Instead of flexible tubes, rigid connector parts can also be present on both sides or on one side.

In contrast to the above examples, the recess in which the mirror, the fibre end and any measuring means are arranged is now covered by the lid 30. The lid 30 can be connected to the lower part with a form fit and/or a force fit and/or a materially cohesive fit.

Lid 30 and lower part 31 are preferably made from the same material. At least the lid 30, however, must be made from a material that is transparent for the optical wavelength used. They are preferably made of polyamide or polycarbonate.

As can be seen from FIG. 9, the lower part 31 and the lid 30 form a common inner drainage channel 12′ with a cable channel 120. The drainage line already described above and any measurement lines extend through this drainage channel 12′ as far as the distal head part of the catheter.

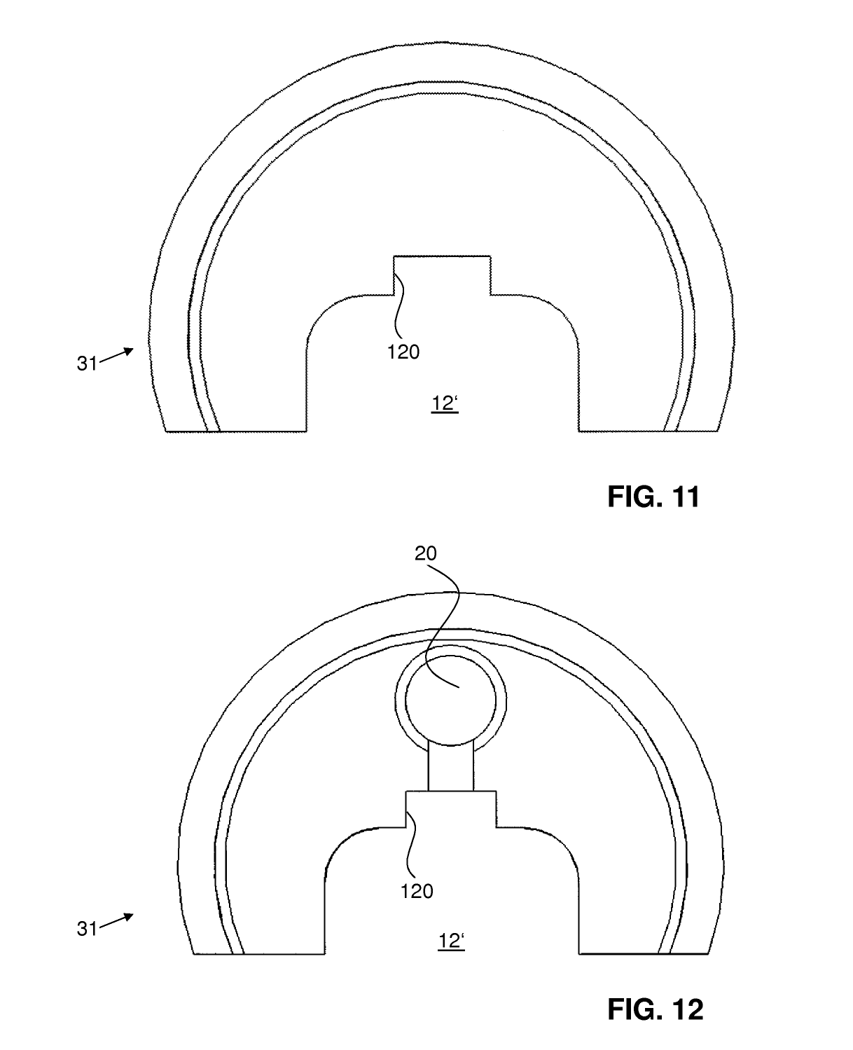

FIG. 11 shows the distal end of the middle piece 3 with the drainage channel 12′. FIG. 12 shows the inner proximal end of the middle piece 3 not visible in FIG. 9. A light conductor channel 20 extends parallel to the drainage channel 12′ but spaced apart therefrom. Here too, it is preferable to use exactly one optical fibre as light conductor. However, there can also be up to five optical fibres, in particular two or three.

Under the aperture 16, the drainage channel 12′ extends along the entire length of the middle piece 3.

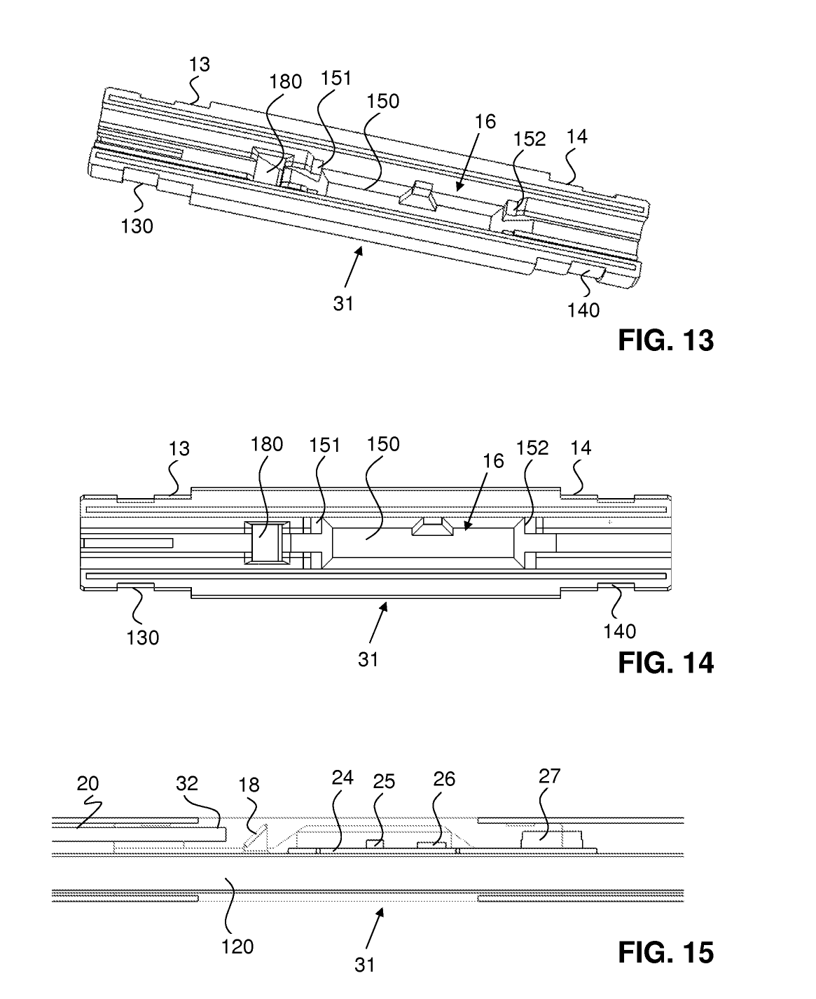

FIGS. 13 to 15 show the lower part 31 in detail. It has the aperture 16. A receiving area 150 for the electronics is present in this aperture 16. Proximal and distal receiving surfaces 151, 152 are formed integrally on both end areas of the receiving area 150. A circuit board 24 can be secured, for example adhesively affixed or screwed, onto these plane receiving surfaces 151, 152.

A photodetector 25 can be secured on the circuit board 24. It is also possible for a temperature sensor 26 and/or a pressure sensor 27 to be present. However, these sensors can also be arranged at another location. As has already been mentioned above with reference to the other examples, the pressure sensor is preferably arranged in the tip element 1.

An inclined surface 180 is present outside this receiving area 150. This inclined surface 180 can itself be designed as a reflection surface. However, it preferably serves as a holder for a mirror 18. The mirror 18 in this example is a plane-parallel mirror, such that the inclined surface 180 is also designed as a plane surface. The inclined surface 180 is preferably oriented at a 45° angle to the longitudinal direction of the middle piece 3.

The light conductor channel 20 extends in the lower part 31 and ends adjacent to the inclined surface 180. The light conductor 32 is shown in FIG. 15. It will be seen that it ends before the mirror 18, such that light emitted from the light conductor 32 is deflected from the mirror 18 and conveyed radially outward.

In this example, light reflected in the tissue is no longer fed back via the light conductor and evaluated. It is instead detected by the photodetector, and an electrical signal is transferred to an external evaluation unit via a signal line. The cables necessary for this purpose run inside the cable channel 120.

This middle piece 3 can be used with one of the tip elements 1 described above. However, it can also be used with other tip elements 1.

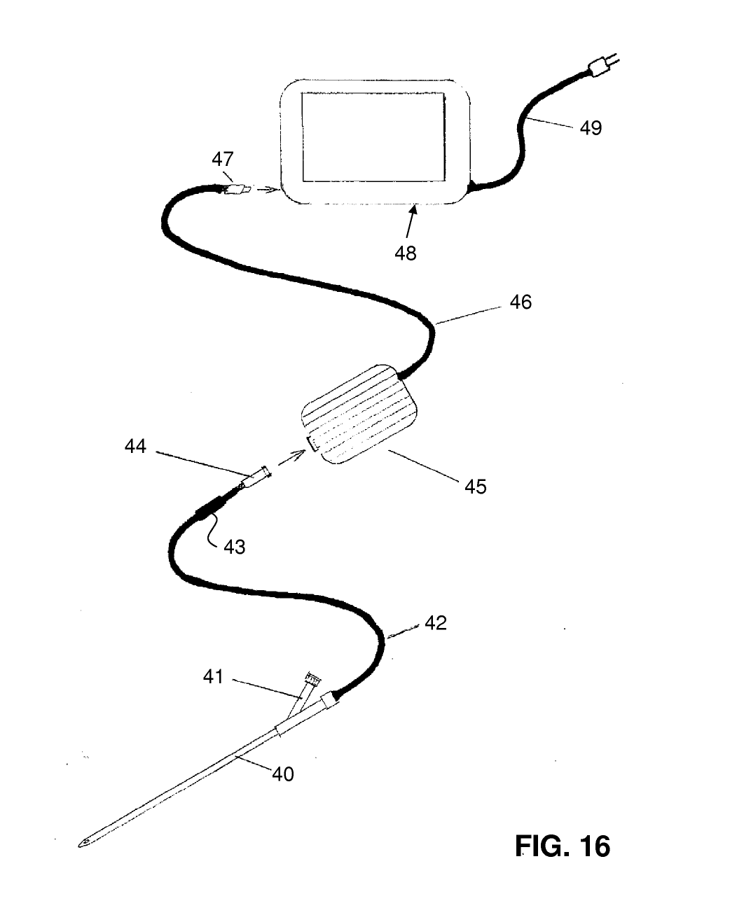

FIG. 16 shows the overall system. The catheter or the probe 40 is provided at the end of the drainage channel with an y-connector with a Luer lock. A first line 42, which comprises electrical conductors and also at least one optical conductor, is connected with a distal end to the probe 40. In the area of the proximal end, it has a calibration unit 43, and it is provided at this end with a first plug 44. This plug 44 permits a connection to a portable electronics unit 45. This electronics unit 45 preferably comprises a potential insulator, an A/D converter, a pre-amplifier, and a light source for coupling light into the optical conductor that leads to the middle piece 3 of the catheter.

The portable electronics unit 45 is to an evaluation unit 48 via a second, purely electrical line 46, which in turn is provided with a second plug 47. A third line represents the mains supply cable. However, the evaluation unit 48 can of course also be battery-operated.

The advantage of this overall system is that the electronics unit 45 is relatively close to the patient. The sensitive optical lines can therefore be made relatively short.

In the examples described above, an optical glass fibre is preferably used in the near infrared range. Its diameter is preferably approximately 0.6 mm inclusive of its sheath and approximately 0.2 mm without its sheath. The length of the middle piece 3 is preferably 20 to 30 mm, and its diameter is preferably 3 mm. The mirror surface preferably measures 1×1 mm. The drainage channel preferably has a diameter of 0.9 to 1.5 mm, preferably 1.0 or 1.2 mm. The width of the circuit board is preferably 1.4 mm. A 9-pole or 10-pole cable, which extends as far as the plug 44, is normally used inside the middle piece 3.

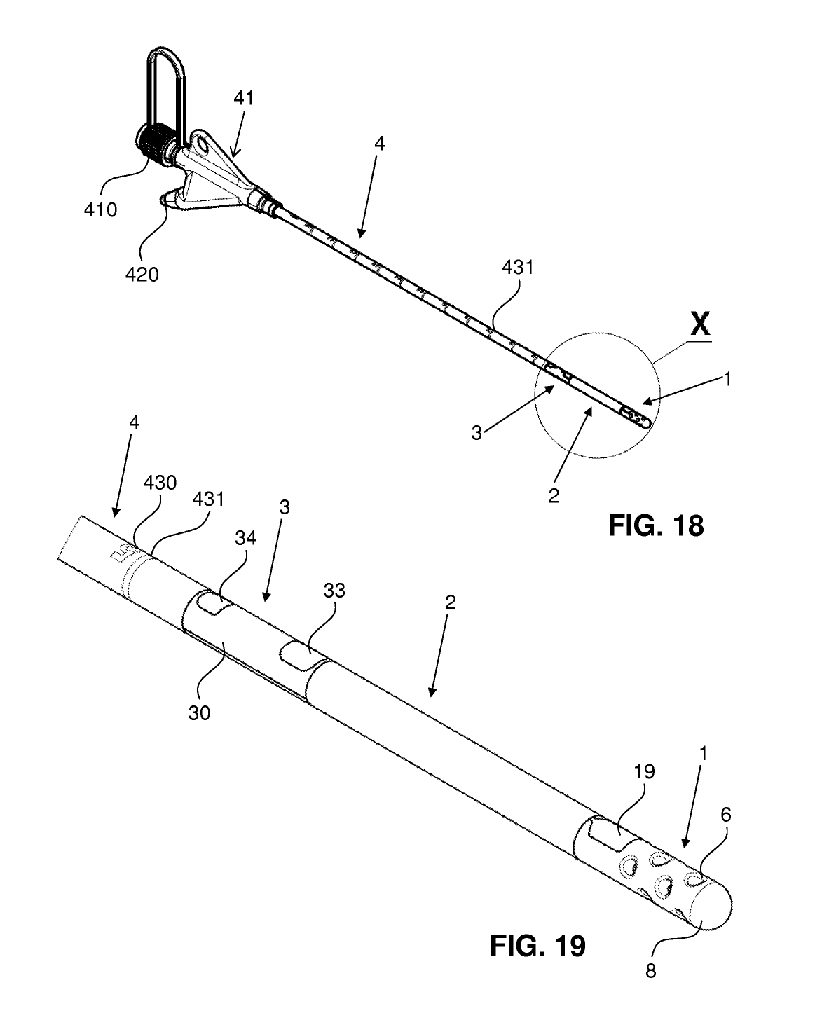

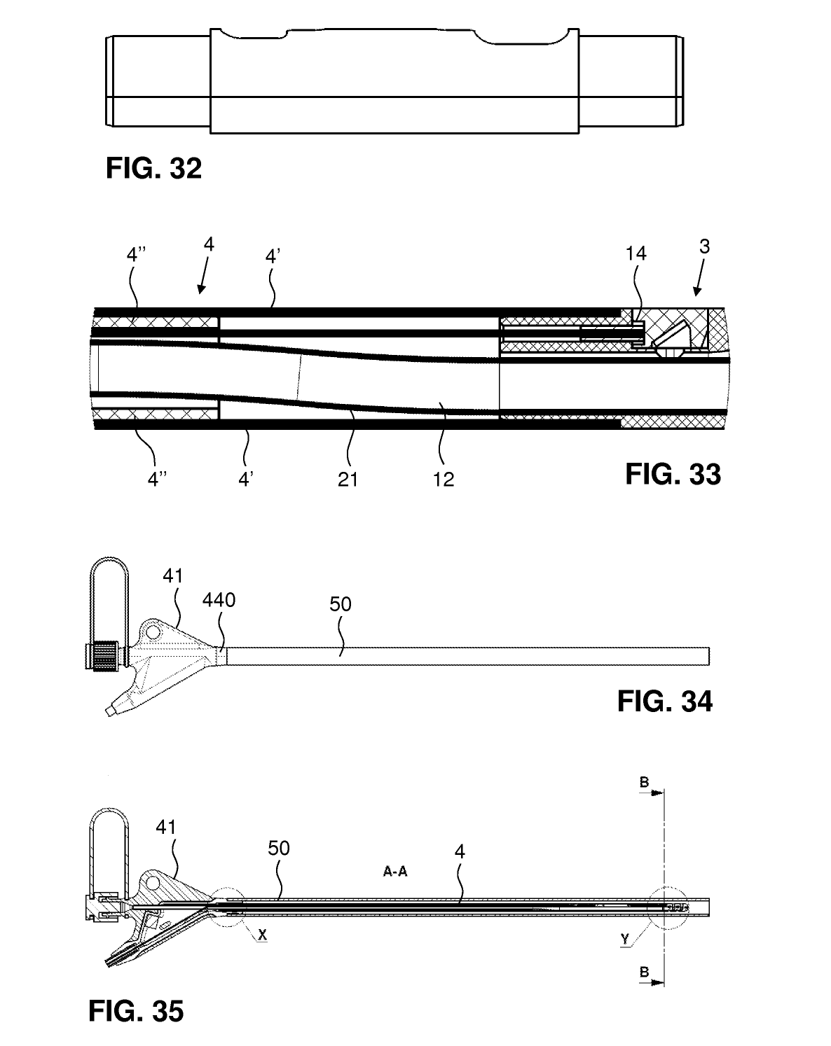

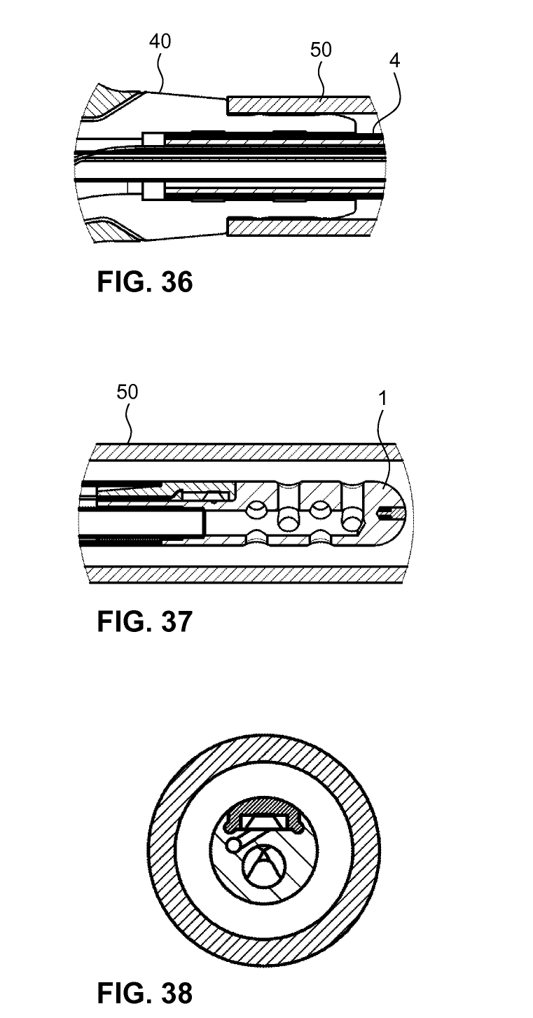

FIGS. 18 to 40 show a second embodiment of the inventive measurement device. Same elements have the same reference numbers.

The measurement device comprises, as shown in FIG. 18, a tip 1 as described above, the first tube part 2, the middle piece 3 and the second tube part 4 ending in the y-connector 41. The y-connector 41 comprises two or more ports 420, wherein at least one of them is closed by a cap 410.

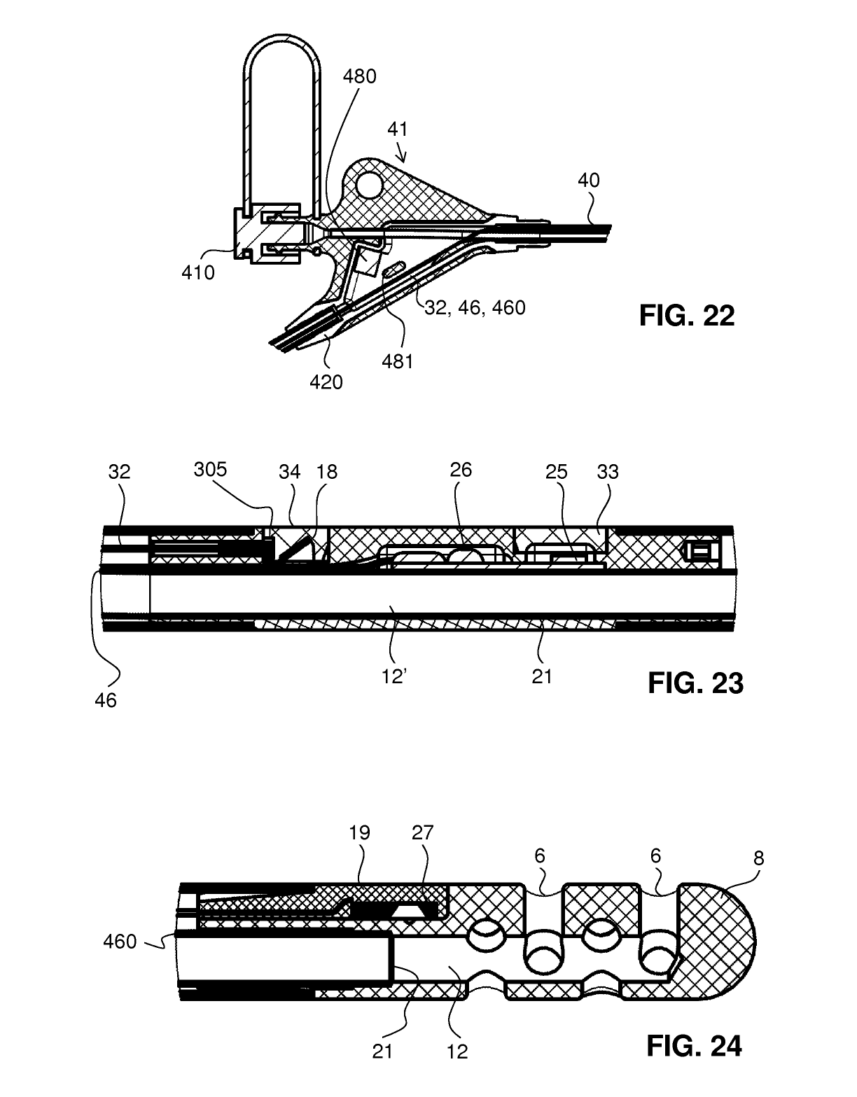

As can be seen in FIG. 22, the light conductor 32, the purely electronic line 46 and a pressure sensor line 460 extend through the open port 420, wherein the closed other port is used for introducing guide wires and/or for fluid transfer.

As also shown in FIG. 22, the y-connector 41 may comprise a data chip 480, such as an EEPROM, which identifies the device. For example, it can comprise information about the sensors, calibration data, date about the first use of the device and manufacturing data. Reference number 481 refers to a mounting aid for the light conductor 32, the second line 46 and the pressure sensor line 46″. It is for example a nose protruding into the inner space of the y-connector 41.

The second tube part 4 preferably consists of two tubes, an inner second tube 4′ and an outer second tube 4″. This can be seen in FIG. 33. The inner second tube 4′ has a slightly smaller diameter than the outer second tube 4″. They are preferably bonded to each other along at least a part of their length.

The distal end of the outer second tube 4″ protrudes the distal end of the inner second tube 4′ so that only the outer second tube 4″ extends over the proximal fastening piece 14 of the middle piece 3 and is connected to it. The inner second tube 4′ ends at a distance to the proximal fastening piece 14. This reduces the outer diameter of the device wherein the second tube part 4 has nevertheless a sufficient thickness to ensure stability of the tube when introduced into the patient. At the proximal end of the second tube part 4 the two ends of the inner and outer second tube 4′, 4″ preferably end in a common plane. In other embodiments, they end in a step as well.

The first tube part 2 may be likewise made of an inner and an outer tube, the outer tube protruding the inner tube at least at the proximal end, preferably also at the distal end. In this embodiment however, the first tube part 2 is shaped like the well-known catheter tubes.

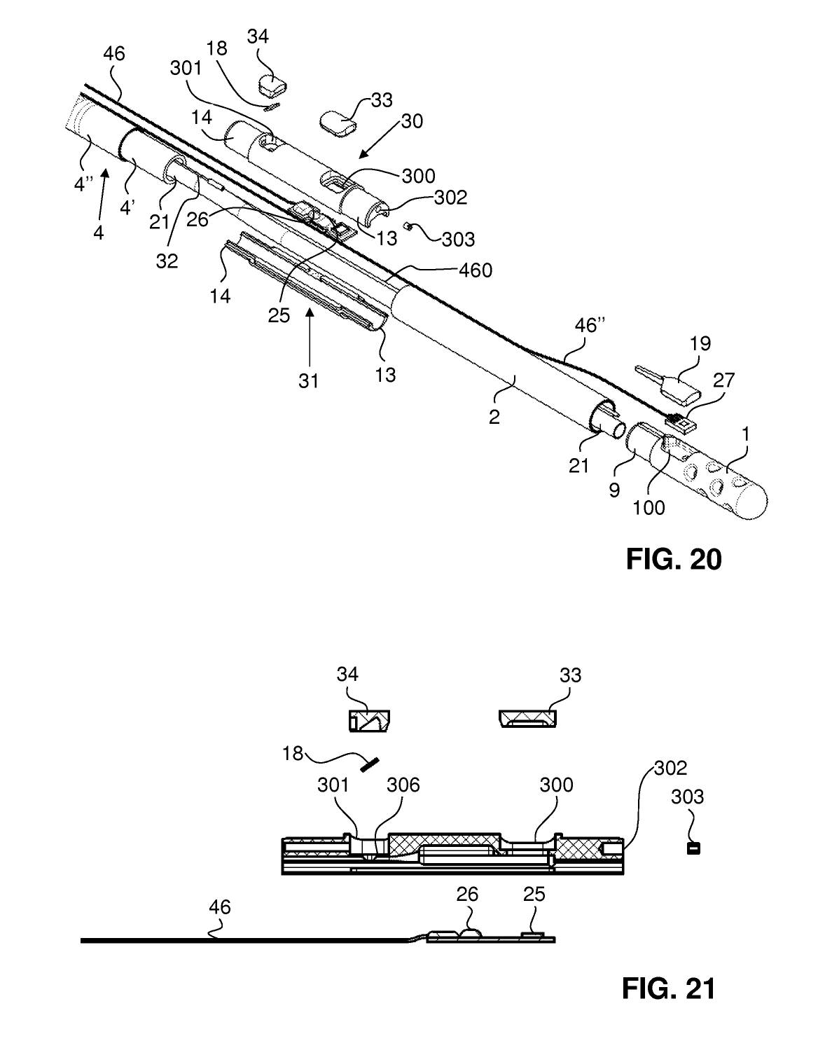

Note is taken that FIG. 20 shows the inner second tube 4′ in a protruding state. This is for better view only and should not be in contradiction to the above mentioned description. In addition, the electronic lines and the fiber optic, i.e. the light conductor, extend outside of the first and second tube parts 2, 4, which is for better view as well. In the assembled device, they extend inside these tube parts 2, 4.

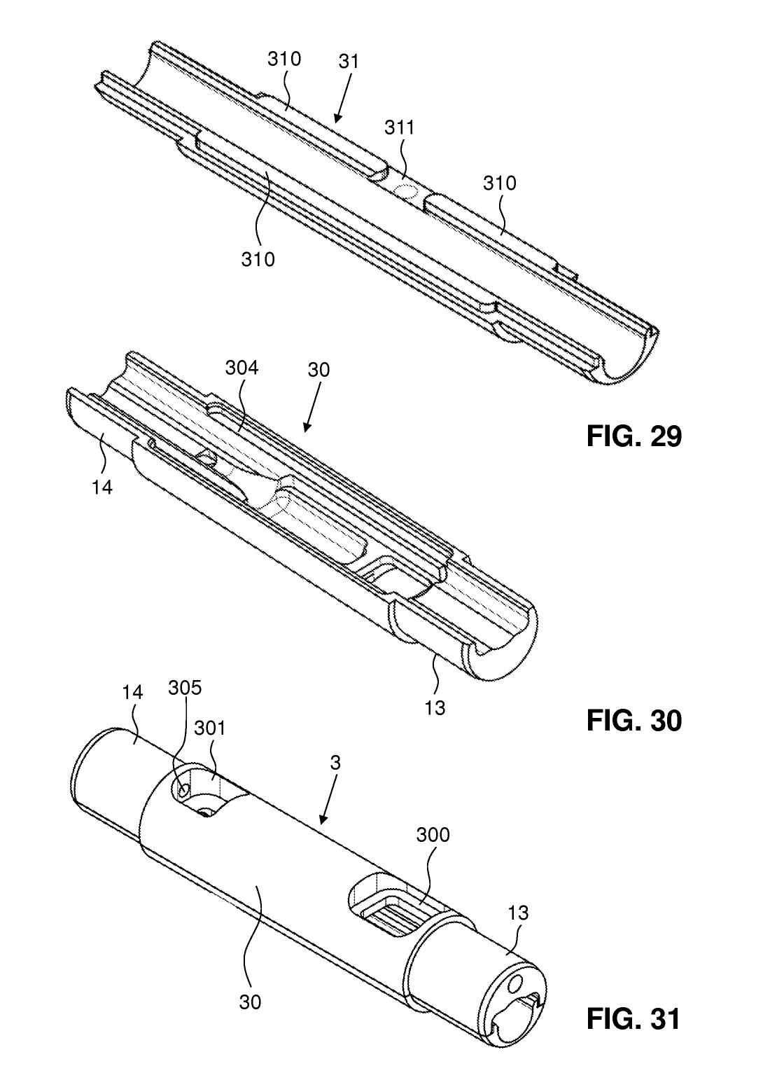

The middle piece 3 of this embodiment comprises a lid or an upper part 30 and a lower part 31 as can be best seen in FIGS. 29 to 32. The two parts 30, 31 are preferably formed as half tubes which can be fixed to each other so that they form together a tube. As shown in FIG. 30, the upper part 30 comprises a notch 304 on both sides of this diameter. The lower part 31 as shown in FIG. 29 comprises a corresponding adjustment longitudinal nose 310 on both sides which engages this notch 304.

The middle piece 3 comprises the distal fastening piece 13 and the proximal fastening piece 14 for connection with the first tube part 2 and the second tube part 4 respectively. In the embodiment shown, an X-ray marker 303 is preferably placed in a hole 302 of the distal fastening piece 13, as shown in FIG. 20. In other embodiments, the X-ray marker is preferably arranged in the tip element 1 and more preferably at its distal end.

The inner side of the lower part 31 is preferably smooth and comprises not recesses or protrusions.

The upper part 30 of the middle piece 3 comprises two openings or windows 300, 301 which form two parts of a recess. The distal first window 300 is for receiving signals from the patient, the proximal second window 301 for emitting light into the patient. The first window 300 is closed with a sensor cover 33, the second window 301 is closed with a mirror holder 34 as shown in FIGS. 19 to 21.

In other embodiments, the light source is directly placed in this second window 301 and no light conductor is present. In this case, the light source is preferably an LED or a laser chip, preferably a bare die.

The sensor cover 33 and the mirror holder 34 are preferably transparent for the at least one wavelength used in this device. Preferably they are made of polycarbonate. Preferably a wavelength of 808 nm and/or 905 nm is used. Other wavelength may be used as well.

The lower part 31 is preferably transparent as well so that it can be bonded to the upper part 30 more easily using UV light. Also the fixation of the elements arranged within the middle piece 3 can be established more easily when the lower part is transparent. Preferably the lower part is not only transparent for UV light but also for visible light so that elements arranged within the part are visible when mounted and fixed.

Preferably, the upper part 30 is made of none-transparent material. Preferably, it is made of PEEK (polyether ether ketone). A none-transparent upper part 30 has the advantage, that scattered or reflected light can only reach the photodetector 25 through the first window 300.

The upper part 30 comprises a channel with an outlet opening 305 directed to the second window 301. The light conductor 32 extends to this opening so that light can be guided along the longitudinal axis of the device to a mirror 18 arranged in the second window. The mirror 18 is fixed in the window. The window holder 34 holds the mirror 18 in a specific angle relative to the longitudinal axis of the device. A mounting hole 306 shown in FIG. 21 arranged at the bottom of the second window 301 helps to mount the mirror 18 at the predetermined place and angle.

A sensor plate is arranged below the first window 300. This sensor plate comprises at least the photodetector 25, but preferably also the temperature sensor 26 and/or other sensors. In a first variant, only one photodetector 25 is present. In other variants, more than one photodetector is present, especially when more than one wavelength is used.

As can be seen in FIG. 23, the at least one photodetector 25 is arranged in the first window 300 and covered by the sensor cover 33.

Using two windows instead of only one has the advantage, that the accuracy of the measurement is improved since no light can be forwarded within the middle piece 3 from the light conductor to the photodetector. This arrangement optimizes the optical path.

The temperature sensor 26 is preferably arranged under the none-transparent region of the upper part 30 between the first and the second window 300, 301. Preferably a diode is used as a temperature sensor. Since it is arranged in an area where no light can enter, the temperature measurement is not disturbed by emitted or reflected light.

As shown in FIGS. 19, 20 and 24, the pressure sensor 27 is arranged in the distal tip element 1. Preferably it is a sensor chip and more preferably a bare die. In this embodiment, it is arranged at the proximal side of the drainage outlets of the tip element 1. In other embodiments it is arranged at the distal end of the tip element, i.e. in front of the drainage openings.

The tip element 1 comprises a cavity 100 being closed with a sealing element 19, such as a sealing compound made of silicone.

The pressure sensor 27 is arranged in this cavity 100, wherein the electronic pressure sensor line 46″ extends from the sensor 27 along the device to the y-connector 41.

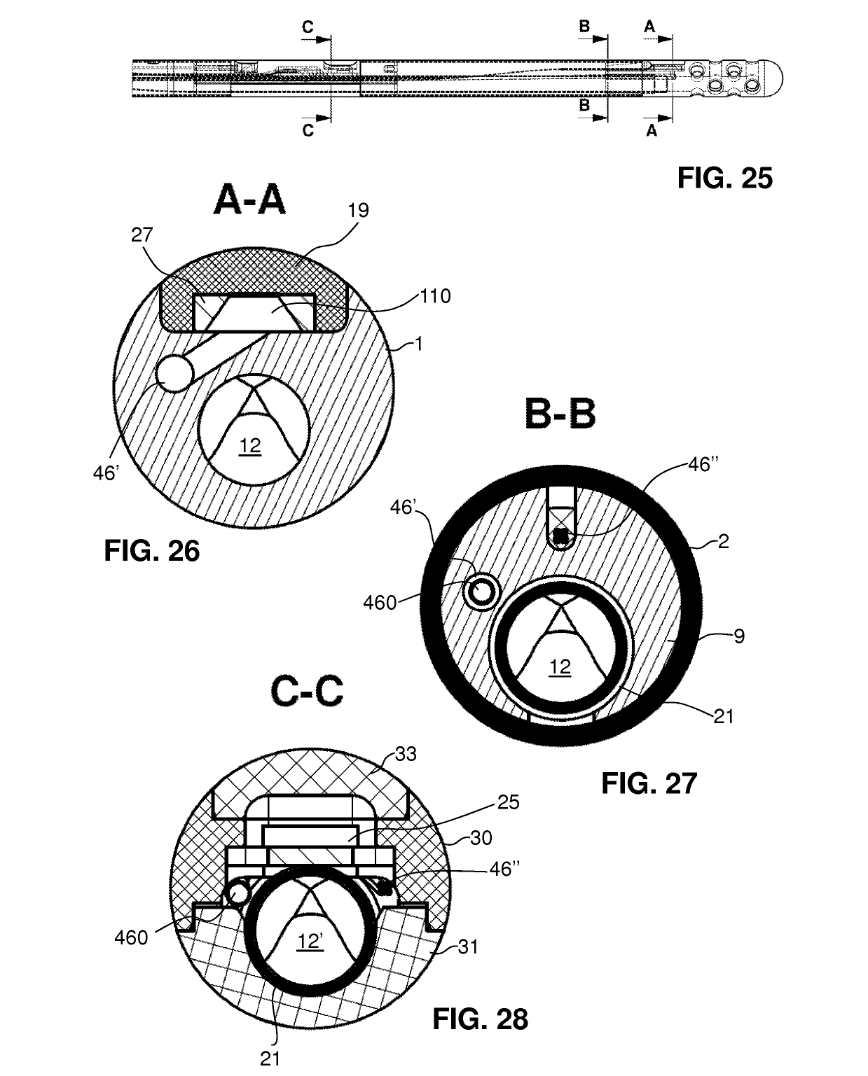

FIGS. 26 to 28 show cross-sections along the device, as shown in FIG. 25. In FIG. 26, the distal tip element 1 with the drainage channel 12 can be seen as well as the cavity 100 closed with the sealing element 19. As can be seen, a space 110 is present under the pressure sensor 27. The space 110 is connected to a pressure equalization channel 46′ which extends to the outside of the device, so that the sensor 27 is exposed to ambient pressure. In other embodiments, the pressure sensor is an absolute pressure sensor or a fiber-optic sensor.

In FIG. 27, the distal end of the first tube part 2 is arranged over and bonded to the fastening piece 9 of the tip element 1, as can also be seen in FIGS. 20 and 24.

In FIG. 27, the pressure equalization channel 46′ and the pressure sensor line 46″ can be seen. The pressure equalization channel 46′ formed within the tip element 1 changes now into a pressure equalization tube 460 extending along the longitudinal axis of the device.

FIG. 28 shows the first sensor cover 33 arranged over the photodetector 25, the pressure sensor line 46″ and the pressure equalization tube 460 as well as the inside tube 21.

FIGS. 34 to 38 show an optional feature for the inventive measurement devices described above. The inventive measurement device is usually adjusted to zero shortly before use. It may also be calibrated as well. In the state of the art, such measurement devices are therefore immersed into water or another appropriate liquid. Since pressure sensors or also other sensors are light sensitive, the water reservoir has to be none-transparent for the relevant wavelengths. Usually it is black so that no visible light can reach the sensor during zero adjustment. It may also be non-transparent for other or only for other wavelength, depending on the sensibility of the pressure sensor to a specific wavelength.

It is an additional inventive idea to store the measurement device in a none-transparent protection sleeve 50 covering at least the sensor parts of the device. The protection sleeve is at least none-transparent for the relevant wavelengths, i.e. the wavelengths which the sensor is sensible of. Preferably, the protection sleeve is black.

Preferably the sleeve 50 is pushed over the distal end of the y-connector 41 and protrudes the distal end of the tip element 1. It is arranged at a distance to the tubes 2, 4, the middle piece 3 and the tip element 1, as can be seen in FIGS. 35 to 38. This sleeve serves as protection when the device is transported.

For zero adjustment or calibration, the measurement device can be immersed together with the protection sleeve 50 in any kind of container, since no light can reach the sensor in any case. The sleeve 50 is only be removed from the measurement device shortly before the device is actually introduced into the patient. This simplifies the handling of the device and reduces mishandling.

Note is made that the embodiment according to FIGS. 18 to 38 can be combined and varied following the teaching and disclosure of the previously described embodiments as well.

### LIST OF REFERENCE NUMBERS

1 tip element
100 cavity
110 space
2 first tube part
3 middle piece
30 upper part/lid
300 first window
301 second window
302 hole
303 marker
304 notch
305 outlet opening
306 mounting hole
31 lower part
310 adjustment nose
311 gap
32 light conductor
33 sensor cover
34 mirror holder
4 second tube part
4′ inner second tube
4″ outer second tube
5 elongate opening
6 round opening
8 tip
9 fastening piece
10, 10′ pressure sensor channel
11 web
12, 12′ drainage channel
120 cable channel
13 fastening piece, distal
130 distal adhesive groove
14 fastening piece, proximal
140 proximal adhesive groove
15 middle area
150 receiving area for electronics
151 proximal receiving surface
152 distal receiving surface
16, 16′ recess, indentation, aperture
17 light exit surface
18 reflection surface/mirror
180 inclined surface
19 sealing element
20 light conductor channel
21 inside tube
22 temperature measurement channel
23 inner surface with light exit surface area and reflection surface area
24 circuit board
25 photodetector
26 temperature sensor
27 pressure sensor
40 probe
430 marking number
431 marking ring
440 stop collar
41 y-connector
410 cap
420 port
42 first line (optoelectronic)
43 calibration
44 first plug
45 portable electronics unit
46 second line (purely electronic)
46′ pressure equalization channel
46″ pressure sensor line
460 pressure equalization tube
47 second plug
48 evaluation unit
480 data chip (EEPROM)
481 mounting guide
49 third line
50 protection sleeve

1. A catheter for measuring a flow of blood through a body tissue, wherein a light emitter for emitting light into the body tissue and at least one light receiver for receiving light reflected in the body tissue and back to the catheter are arranged in the catheter, wherein the catheter comprises a middle piece and a first and a second connection tube, wherein the middle piece has a distal fastening piece for fastening to the first connection tube and a proximal fastening piece for fastening to the second connection tube, and wherein the light emitter and the at least one light receiver are arranged in the middle piece.

2. The catheter according to claim 1, wherein the middle piece is rigid.

3. The catheter according to claim 1, wherein the catheter further comprises a rigid head part, wherein the head part has a third fastening piece for fastening to the first connection tube, such that the head part is arranged at a distance from the middle piece.

4. The catheter according to claim 1, wherein at least the first connection tube is flexible.

5. The catheter according to claim 1, wherein the middle piece is made of metal or plastic.

6. The catheter according to claim 1, wherein the middle piece comprises at least a first and a second window, wherein the light emitted from the light emitter is emitted through the second window into the body tissue and the light reflected in the body tissue is received by the at least one light receiver through the first window.

7. The catheter according to claim 6, wherein the first and the second window are arranged on the same side of the middle piece.

8. The catheter according to claim 6, wherein the middle piece comprises an upper part and a lower part being connected to each other, wherein the at least first and second window are arranged in the upper part.

9. The catheter according to claim 8, wherein the upper part is not transparent for the wavelength used.

10. The catheter according to claim 8, wherein the lower part is transparent at least for a wavelength used during assembly of the lower and upper part.

11. The catheter according to claim 10, wherein the lower part is transparent for visible light and/or for UV light.

12. The catheter according to claim 6, wherein a temperature sensor is arranged in the middle part between the first and the second window.

13. The catheter according to claim 12, wherein the temperature sensor is a diode.

14. The catheter according to claim 1, wherein the second connection tube comprises an inner tube having a distal end and an outer tube having a distal end, wherein the distal end of the outer tube protrudes the distal end of the inner tube and wherein the second tube is attached to the proximal fastening piece with the outer tube only.

15. The catheter according to claim 14, wherein the inner tube is bonded to the outer tube.

16. The catheter according to claim 1, wherein the light emitter is an optical conductor that has a distal end, and wherein the distal end is held in the middle piece.

17. The catheter according to claim 1, wherein a pressure sensor and/or a temperature sensor are arranged in the middle piece or in the tip element.

18. The catheter according to claim 1, wherein the at least one light receiver is a photodetector arranged in the middle piece.

19. The catheter according to claim 18, wherein a receiver in which a circuit board is secured is present in the recess, and wherein at least the photodetector is arranged on the circuit board.

20. The catheter according to claim 1, wherein a mirror is present in the middle piece, lies opposite a light exit surface area of the light emitter and deflects light issuing from the light emitter into the body tissue.

21. The catheter according to claim 1, wherein it has a drainage channel extending through the middle piece.

22. The catheter according to claim 1, wherein the catheter has a smooth and unchanging diameter at least along the first tube, the middle piece and the second tube.

23. A catheter system with a catheter and a protection sleeve covering the catheter along its length, wherein the protection sleeve comprises a distal end which protrudes a distal end of the catheter, wherein the protection sleeve is open at its distal end and extends at a distance to the catheter located within the sleeve and wherein the protection sleeve is not transparent at least for visible light.

24. The catheter system according to claim 23, wherein the catheter is a catheter according to claim 1.

25. A catheter comprising a tube made of a flexible material and a stiff element attached to the tube, wherein the tube comprises an inner tube having a first end and an outer tube having a second end located on the same side as the first end, wherein the first end of the outer tube protrudes the second end of the inner tube, wherein the tube is attached to the stiff element with the outer tube only.

26. The catheter according to claim 25, wherein the inner tube is bonded to the outer tube.

27. The catheter according to claim 25, wherein the inner tube is arranged concentrically in the outer tube.

28. A tube of a catheter according to claim 25, wherein the tube comprises an inner tube having a first end and an outer tube having a second end located on the same side as the first end, wherein the first end of the outer tube protrudes the second end of the inner tube, wherein the tube is attached to a stiff element with the outer tube only.

29. A catheter for measuring a flow of blood through a body tissue, wherein a light emitter for emitting light into the body tissue and at least one light receiver for receiving light reflected in the body tissue and back to the catheter are arranged in the catheter, wherein the catheter comprises a connection tube and a stiff element, wherein the light emitter and the at least one light receiver are arranged in the stiff element, wherein the stiff element comprises at least a first and a second window, wherein the light emitted from the light emitter is emitted through the second window into the body tissue and the light reflected in the body tissue is received by the at least one light receiver through the first window, wherein the stiff element comprises an upper part and a lower part being connected to each other, wherein the at least first and second window are arranged in the upper part, wherein the upper part is not transparent for the wavelength used.

30. The catheter according to claim 29, wherein the lower part is transparent at least for a wavelength used during assembly of the lower and upper part.
